(19) United States
(12) Patent Application Publication
(10) Pub. No.: US 2017/0056232 A1
(43) Pub. Date: Mar. 2, 2017

(54) Arm Support Device

(71) Applicant: David Ka Wai Yeung, Fotan (HK)

(72) Inventor: David Ka Wai Yeung, Fotan (HK)

(21) Appl. No.: 15/248,305

(22) Filed: Aug. 26, 2016

Related U.S. Application Data
(60) Provisional application No. 62/210,536, filed on Aug. 27, 2015.

(51) Int. Cl. A61F 5/01

(52) U.S. Cl. CPC A61F 5/0118

(57) ABSTRACT

An arm support device for improving body posture when viewing an object such as when using a smartphone or reading a book. The arm support device generally includes includes a strap which is connected at its first end to the first end of a retainer and at its second end to the second end of the retainer. When so assembled, the strap and retainer form a looped configuration which may be worn around the body of a user. The strap extends around the shoulder and body of the user, with the retainer being positioned to removably and comfortably receive the elbow of the user. By using the arm support device, a user may retain optimal posture to view an object without excess strain on the neck.

### CROSS REFERENCE TO RELATED APPLICATIONS

I hereby claim benefit under Title 35, United States Code, Section 119(e) of U.S. provisional patent application Ser. No. 62/210,536 filed Aug. 27, 2015. The 62/210,536 application is currently pending. The 62/210,536 application is hereby incorporated by reference into this application.

### STATEMENT REGARDING FEDERALLY SPONSORED RESEARCH OR DEVELOPMENT

Not applicable to this application.

### BACKGROUND

Field

Example embodiments in general relate to an arm support device for improving body posture when viewing an object such as when using a smartphone or reading a book.

Related Art

Any discussion of the related art throughout the specification should in no way be considered as an admission that such related art is widely known or forms part of common general knowledge in the field.

Posture is very important when reading a book, viewing an object in one's hand, or using a mobile device such as a smartphone or a tablet. Bad posture can lead to serious issues down the road for an individual as they age. For example, commonly using a head tile of between 15 and 60 degrees when viewing such an object causes a force on the neck of up to 27-60 pounds. This type of repeated, continuous stress on the neck can lead to cervical spine damage, among other ailments.

Prior art solutions aimed at improving posture when viewing object such as reading a book or using mobile electronic devices typically rely on support systems that are attached to a fixed surface. While these types of systems may be suitable for individuals who commonly perform such activities in their own home or office, they do not provide a solution to the numerous individuals who commonly read a book or use mobile devices on-the-go.

### SUMMARY

An example embodiment of the present invention is directed to an arm support device. The arm support device includes a strap which is connected at its first end to the first end of a retainer and at its second end to the second end of the retainer. When so assembled, the strap and retainer form a looped configuration which may be worn around the body of a user. The strap extends around the shoulder and body of the user, with the retainer being positioned to removably and comfortably receive the elbow of the user. By using the arm support device, a user may retain optimal posture to view an object without excess strain on the neck.

There has thus been outlined, rather broadly, some of the features of the arm support device in order that the detailed description thereof may be better understood, and in order that the present contribution to the art may be better appreciated. There are additional features of the arm support device that will be described hereinafter and that will form the subject matter of the claims appended hereto. In this respect, before explaining at least one embodiment of the arm support device in detail, it is to be understood that the arm support device is not limited in its application to the details of construction or to the arrangements of the components set forth in the following description or illustrated in the drawings. The arm support device is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

Example embodiments will become more fully understood from the detailed description given herein below and the accompanying drawings, wherein like elements are represented by like reference characters, which are given by way of illustration only and thus are not limitative of the example embodiments herein.

FIG. 1 is a perspective view of an arm support device in accordance with an example embodiment.

FIG. 2 is a frontal view of an arm support device in accordance with an example embodiment.

FIG. 3 is a top view of an arm support device in accordance with an example embodiment.

FIG. 4 is a bottom view of an arm support device in accordance with an example embodiment.

FIG. 5 is a side view of an arm support device in accordance with an example embodiment being used to view a mobile phone.

FIG. 6 is a perspective view of an arm support device in accordance with an example embodiment being used to view a mobile phone.

FIG. 7 is a frontal view of an arm support device in accordance with an example embodiment being used to view a mobile phone.

FIG. 8 is a perspective view of an arm support device in accordance with an example embodiment being used to read a book.

FIG. 9 is a frontal view of an arm support device which utilizes buckles in accordance with an example embodiment.

FIG. 10 is a frontal view of an arm support device which utilizes hook-and-loop fasteners in accordance with an example embodiment.

### DETAILED DESCRIPTION

### A. Overview.

An example arm support device 10 generally comprises a strap 20 which is connected at its first end 21 to the first end 31 of a retainer 30 and at its second end 24 to the second end 33 of the retainer 30. When so assembled, the strap 20 and retainer 30 form a looped configuration which may be worn around the body of a user 12. The strap 20 extends around the shoulder and body of the user 12, with the retainer 30 being positioned to removably and comfortably receive the elbow of the user 12. By using the arm support device 10, a user 12 may retain optimal posture to view an object 12 without excess strain on the neck.

### B. Straps.

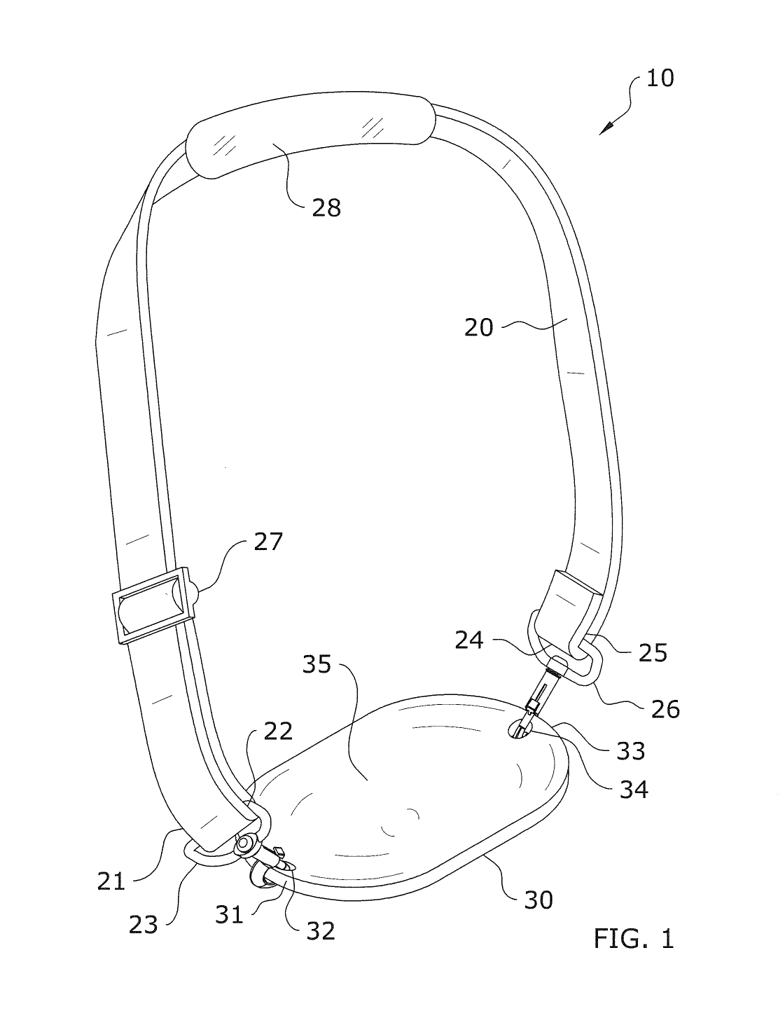

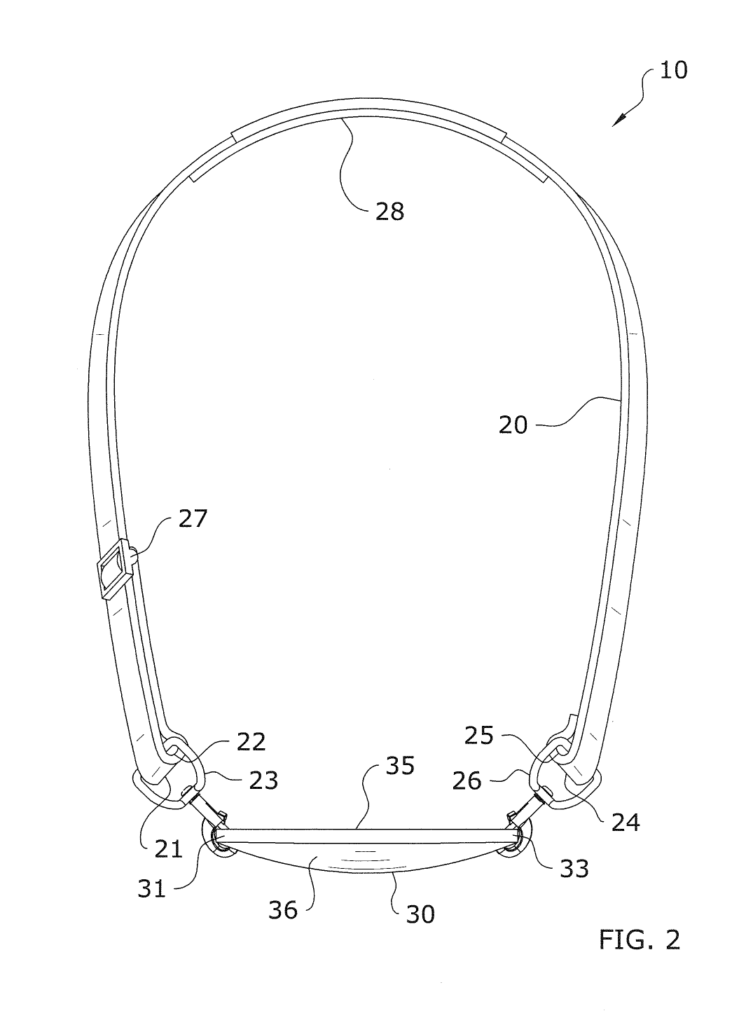

As best shown in FIGS. 1 and 2, the arm support device 10 will generally comprise a strap 20 comprising a first end 21 and a second end 24. The strap 20 will generally comprise an elongated strip of material which, when connected to the retainer 30 such as shown in FIG. 2, will form a looped configuration.

The strap 20 may comprise various materials, sizes, shapes, and configurations, and thus should not be construed as limited by the exemplary figures. For example, the strap 20 may comprise a nylon material. The strap 20 will preferably be comfortable and sufficiently wide to avoid excessive contact pressure on the user 12. The strap 20 may include a non-slip surface to aid in frictional engagement with the user 12 when in use; thus enhancing its stability when resting on the neck and shoulder.

The strap 20 will preferably be adjustable in length to accommodate different user's 12. Given the wide range of heights, weights, and body types of user's 12 of the arm support device 10, the adjustability of the strap 20 will allow for posture to be achieved by any type of user 12. The strap 20 may thus include an adjuster 27 somewhere along its length to allow for the adjustment of the length of the strap 20.

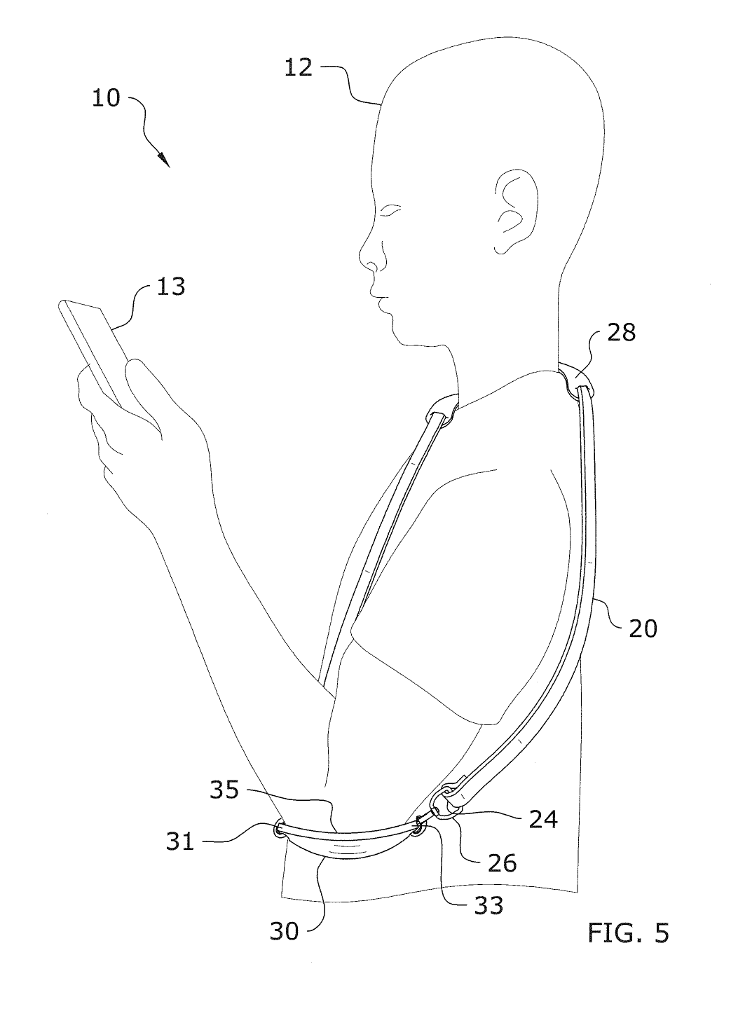

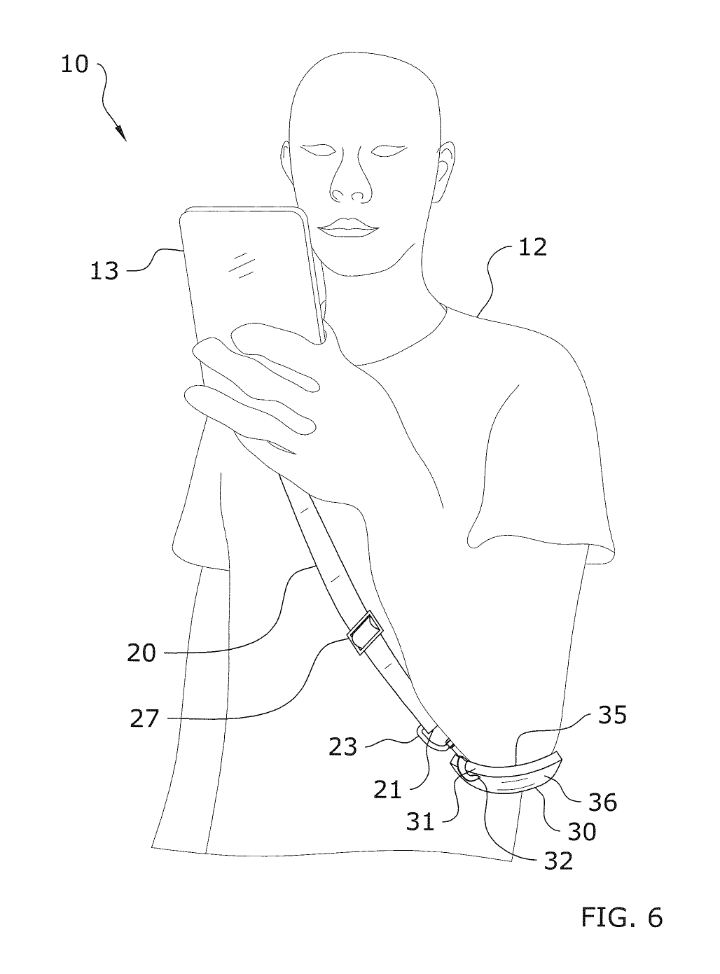

Preferably, the length of the strap 20 will be adjusted by the user 12 such that, when the curled elbow is placed in the retainer 30, the hand-held object 13 is retained at a comfortable eye level with minimized requirement for leaning the head forward. FIG. 5 illustrates an exemplary view of a properly-adjusted strap 20.

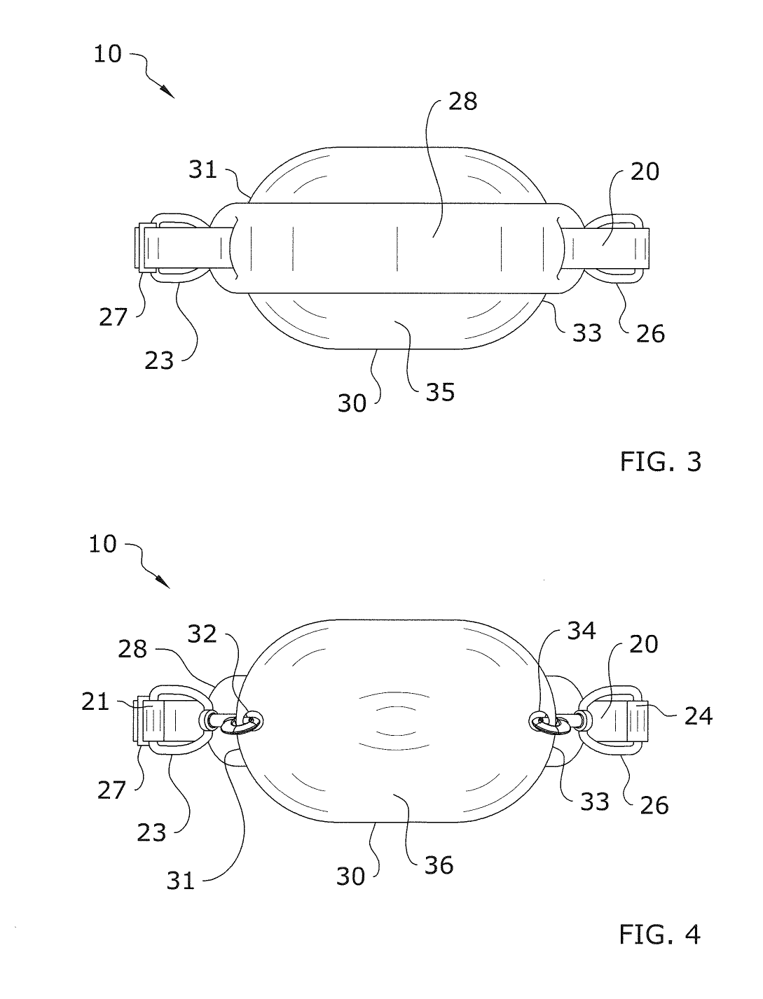

As shown throughout FIGS. 1-4, the strap 20 may include a first loop 22 at its first end 21 and a second loop 25 at its second end 24. A first connector 23 may be connected to the first loop 22 and a second connector 26 may be connected to the second loop 25. The connectors 23, 26 are utilized to connect the respective ends 21, 24 of the strap 20 to the retainer 30 as discussed herein.

While the figures illustrate the connectors 23, 26 being connected, such as removably connected, to the loops 22, 25, it should be appreciated that, in some embodiments, the connectors 23, 26 may be connected directly to the ends 21, 24 of the strap 20 without the need for any loops 22, 25. For example, the connectors 23, 26 could be fastened such as by stitching or other means to the ends 21, 24 of the strap 20 rather than utilizing loops 22, 25 as shown in the exemplary figures.

The connectors 23, 26 allow for the retainer 30 to be removably connected to the strap 20 to form a looped configuration as shown in the figures. It should be appreciated that, in some embodiments, the retainer 30 may be fixedly connected to the strap 20, such as by fasteners including adhesives or stitching. In the embodiments shown in the figures, the retainer 30 is removably connected to the strap 20. This configuration allows for the invention to be easily disassembled for transport or storage when not in use. This configuration also allows for easy support of both right-handed and left-handed users 12.

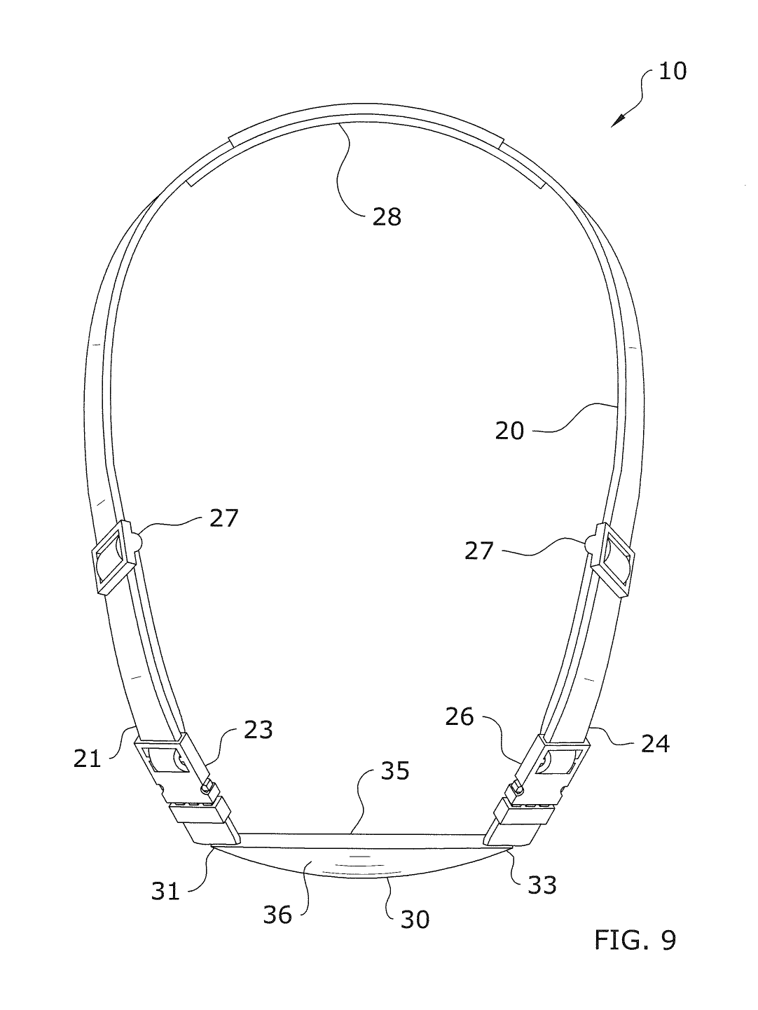

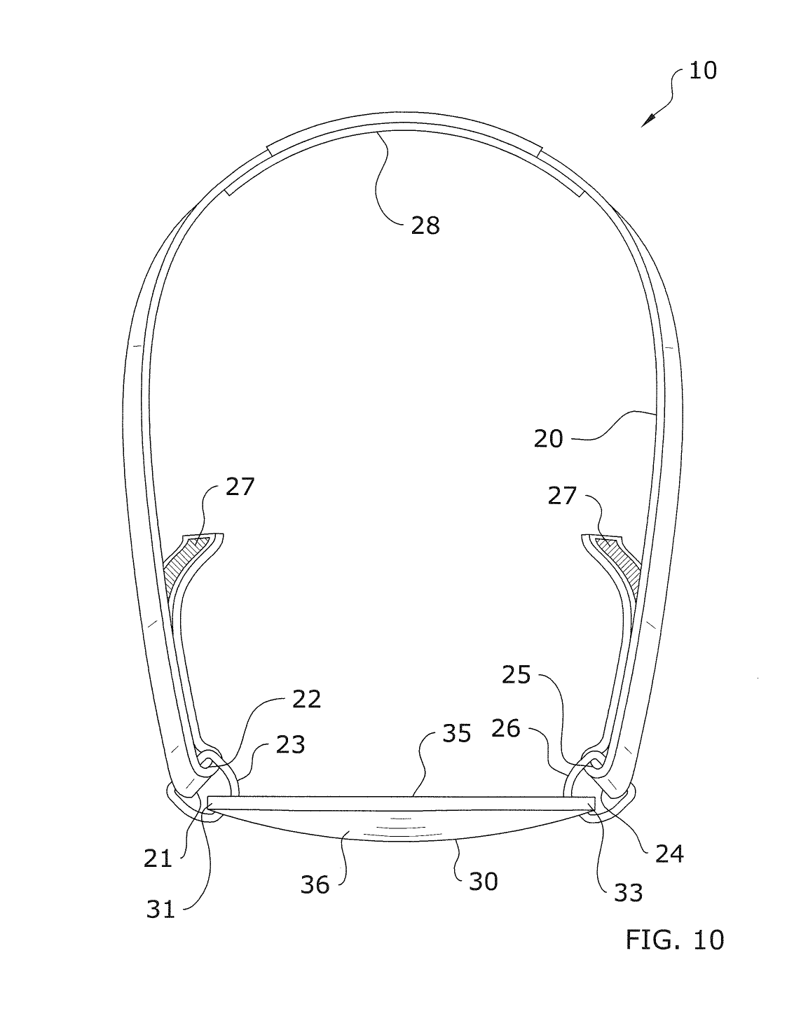

Various types of connectors 23, 26 may be utilized. FIGS. 1-8 illustrate connectors 23, 26 comprising clips that may be removably connected to corresponding receivers 32, 34 on the retainer 30. FIG. 9 illustrates an alternate embodiment in which the connectors 23, 26 comprise buckle connectors and the receivers 32, 34 on the retainer 30 comprise buckle receivers. FIG. 10 illustrates an alternate embodiment in which the connectors 23, 26 comprise a hook-and-loop fastener and loop which are connected to the retainer 30 via receivers 32, 34 comprising openings. It should be appreciated that these are merely exemplary descriptions and illustrations of connectors 23, 26, and that a wide range of other types of connectors 23, 26 could be utilized so long as the strap 20 is easily and removably connected to the retainer 30. The strap 20 may also include a pad 28 to provide comfort to the shoulder of a user 12 when in use.

### C. Retainer.

As shown throughout the figures, a retainer 30 is utilized to support n elbow of a user and thus retainer proper posture for viewing an object 12. The retainer 30 may comprise various structures which comfortably retain the elbow of the user 12 when the present invention is in use to allow for proper posture when viewing an object 12.

As shown in the figures, the retainer 30 may comprise a concave-shaped pouch. The retainer 30 may be circle-shaped, oval-shaped, or other shapes. The retainer 30 may comprise various materials, such as a thick but soft fabric such that the retainer 30 envelopes part of the user's 12 elbow in the flexed position. Other materials may be utilized such as rubber or silicon that has been molded into a concave shape to fit and support the flexed elbow of a user 12. Preferably, the retainer 30 includes a non-slip surface with a concave structure such that the flexed elbow fits securely inside providing stability while holding the object 12.

As best shown in FIGS. 1-4, the retainer 30 may comprise a first end 31 and a second end 33. The first end 21 of the strap 20 may be fixedly or removably connected to the first end 31 of the retainer 30. The second end 24 of the strap 20 may be fixedly or removably connected to the second end 33 of the retainer 30. The retainer 30 may include an inner surface 35 which supports the elbow and an outer surface 36 which faces away from the elbow. The inner surface 35 may comprise a softer material than the rest of the retainer 30 for comfort.

In embodiments in which the strap 20 is removably connected to the retainer 30, the retainer 30 may include receivers 32, 34 adapted to removably engage with corresponding connectors 23, 26 on the strap 20. In the exemplary figures, a first receiver 32 is positioned near the first end 31 of the retainer 30 and a second receiver 34 is positioned near the second end 33 of the retainer 30. The receivers 32, 34 may comprise openings, clips, or the like to which the strap 20 or strap connectors 23, 26 may be removably connected.

### D. Alternate Embodiment.

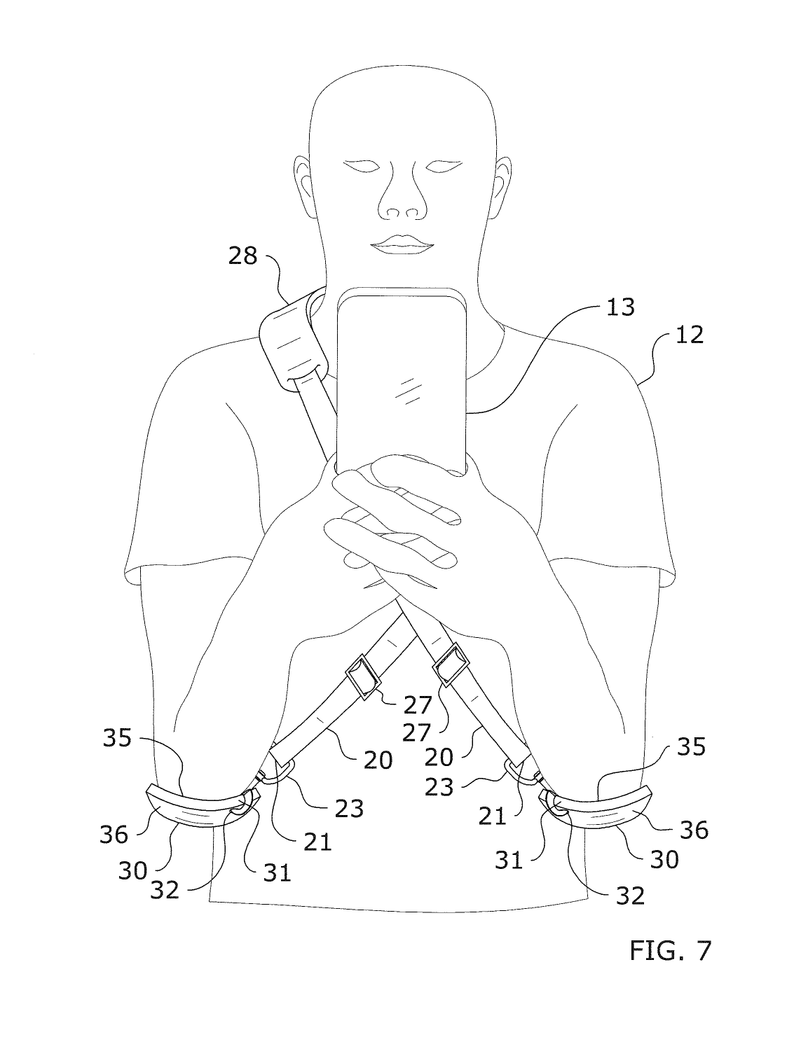

FIG. 7 illustrates an alternate embodiment in which a second retainer 30 is provided to support the second elbow of the user 12. In this embodiment, a second strap 20 extends from a central point on the first strap 20. The second strap 20 may include an adjuster 27 to be adjustable in length. The second strap 20 may also include its own connector 23 for removably connection to the second pouch 30. The second pouch 30 includes its own receiver 32 adapted to removably engage with the connector 23 of the second strap 20. In this manner, both elbows of the user 12 may be supported by its own retainer 30.

### E. Operation of Preferred Embodiment.

FIGS. 5-8 illustrate an exemplary embodiment in use. In use, the retainer 30 is first connected to the strap 20 unless an embodiment with a fixed retainer 30 is being utilized. In embodiments with removable retainers 30, the first end 21 of the strap 20 is connected to the first end 31 of the retainer 30, such as by using a first connector 23 on the strap 20 in combination with a first receiver 32 on the retainer 30. Similarly, the second end 24 of the strap 20 is connected to the second end 33 of the retainer 30, such as by using the second connector 26 and second receiver 34. When so assembled, the strap 20 and retainer 30 will together form a looped structure such as shown in FIGS. 1 and 2.

With the strap 20 and retainer 30 assembled together, the strap 20 may be adjusted such that the retainer 30 is about the same level as the curled elbow of the user 12 at rest. If the left hand is being used to hold the object 13, the left curled elbow is placed in the retainer 30 to provide elbow and arm support. If the right hand is used to hold the object 13, the right curled elbow is placed in the retainer 30 to provide elbow and arm support. The strap 20 extends around the body and shoulder of the user 12. When properly adjusted, the object 13 is close to eye-level with minimal head leaning required to view the object 13.

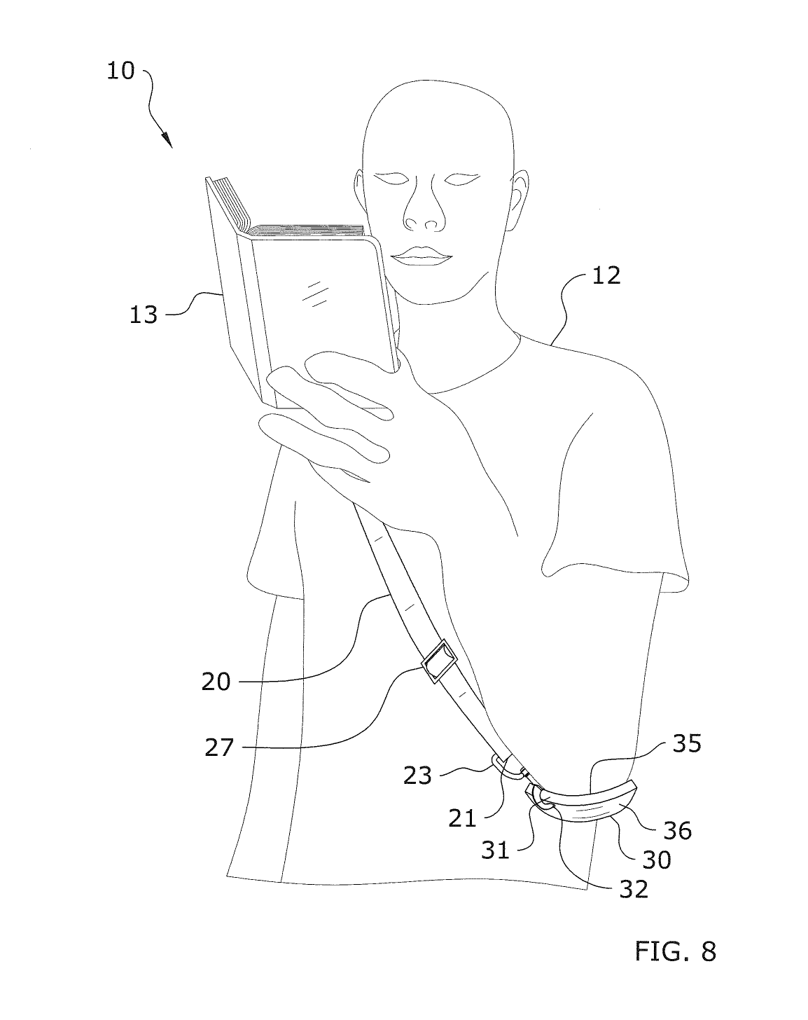

The object 13 may then be viewed with proper posture. Various objects 13 may be utilized. The scope of the present invention should not be construed as limited for use with any particular type of object 13. FIGS. 5-7 illustrate an object 13 comprising a smartphone being used by the user 12. FIG. 8 illustrates an object 13 comprising a book being read by the user 12.

When finished viewing the object 13, the elbow may be easily removed from the retainer 30 by simply stretching the arm outwardly. When stretching the arm, the elbow will naturally be released from the retainer 30 and thus be free to use. This may be important in emergencies to allow one to release the arm from the retainer 30 with a simple arm stretch.

Unless otherwise defined, all technical and scientific terms used herein have the same meaning as commonly understood by one of ordinary skill in the art to which this invention belongs. Although methods and materials similar to or equivalent to those described herein can be used in the practice or testing of the arm support device, suitable methods and materials are described above. All publications, patent applications, patents, and other references mentioned herein are incorporated by reference in their entirety to the extent allowed by applicable law and regulations. The arm support device may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

What is claimed is:

1. An arm support device, comprising:
a strap adapted to be worn around the shoulder of a user, wherein the strap includes a first end and a second end; and
a retainer connected between the first end and the second end of the strap, wherein the retainer is adapted to support an elbow of the user when the user is grasping an object.

2. The arm support device of claim 1, wherein the retainer comprises a pouch.

3. The arm support device of claim 1, wherein the retainer is concave-shaped.

4. The arm support device of claim 1, wherein the strap is length-adjustable.

5. The arm support device of claim 1, wherein the strap includes a shoulder pad.

6. The arm support device of claim 1, wherein the retainer is removably connected to the strap.

7. The arm support device of claim 6, wherein the strap includes a first clip and a second clip for connecting to the retainer.

8. The arm support device of claim 7, wherein the retainer includes a first opening adapted to removably receive the first clip and a second opening adapted to removably receive the second clip.

9. The arm support device of claim 6, wherein the first end of the strap includes a first buckle connector and wherein the second end of the strap includes a second buckle connector.

10. The arm support device of claim 9, wherein the retainer includes a first buckle receiver for removably connecting with the first buckle connector and a second buckle receiver for removably connecting with the second buckle connector.

11. The arm support device of claim 1, wherein the object comprises a book.

12. The arm support device of claim 1, wherein the object comprises a mobile phone.

13. An arm support device, comprising:
a first strap adapted to be worn around the shoulder of a user, wherein the strap includes a first end and a second end;
a second strap extending from the first strap;
a first retainer connected between the first end and the second end of the first strap, wherein the first retainer is adapted to support a first elbow of the user when the user is grasping an object; and
a second retainer connected to a distal end of the second strap, wherein the second retainer is adapted to support a second elbow of the user when the user is grasping an object.

14. The arm support device of claim 13, wherein the first retainer and the second retainer each comprise a concave shape.

15. The arm support device of claim 13, wherein the first retainer and the second retainer each comprise a pouch.

16. The arm support device of claim 13, wherein the object comprises a book.

17. A method of providing support when viewing an object, comprising:
extending a strap around a shoulder and body of a user, wherein the strap comprises a retainer for providing support to an elbow of the user;
adjusting a length of the strap such that the retainer is at the same level as the elbow of the user when the elbow is at rest;
positioning the elbow of the user within the retainer such that the retainer supports the elbow; and
viewing the object at eye-level of the user.

18. The method of claim 17, wherein the object comprises a mobile phone.

19. The method of claim 17, further comprising the step of removing the elbow from the retainer by stretching an arm of the user outwardly.

20. The method of claim 17, wherein the retainer is removably connected to the strap.
